(19) United States
(12) Patent Application Publication
(10) Pub. No.: US 2006/0004274 A1
(43) Pub. Date: Jan. 5, 2006

(54) Fusing nuclear medical images with a second imaging modality

(76) Inventor: Eric G. Hawman, Schaumburg, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/881,599

(22) Filed: Jun. 30, 2004

(51) Int. Cl. A61B 5/05

(52) U.S. Cl. 600/407

(57) ABSTRACT

Co-registration or fusion of nuclear medical images with images of the same region obtained with a different modality, such as Computerized Tomography (CT), Magnetic Resonance Imaging (MRI) and Ultrasound (US) is improved by using the Compton scatter principle to enhance anatomical boundary secondary image information in nuclear image data that is optimized for imaging function. The alignment of the primary nuclear image data with image data of the second modality is facilitated through use of a geometric transform obtained by co-registering the Compton scatter image with the second modality image.

### BACKGROUND OF THE INVENTION

1. Field of the Invention

This invention relates generally to nuclear medicine, and more specifically relates to co-registration or fusion of nuclear medical images with images of the same region obtained with a different modality, such as Computerized Tomography (CT), Magnetic Resonance Imaging (MRI) and Ultrasound (US). The invention uses the Compton scatter principle to enhance anatomical boundary secondary image information in nuclear image data to facilitate the alignment of primary nuclear image data with image data of the second modality.

2. Background and Prior Art

In nuclear imaging, a patient is injected with or swallows a radioactive isotope which has an affinity for a particular organ, structure or tissue of the body. In single photon nuclear imaging, either planar or tomographic (SPECT), gamma rays are then emitted from the body part of interest, are collimated by a collimator so that only gamma photons traveling in a direction nearly perpendicular to the surface of a detector head are allowed to impinge on the detector head, and are detected by a gamma camera apparatus including the detector head, which forms an image of the organ based on the detected concentration and distribution of the radioactive isotope within the body part of interest.

In positron emission tomography (PET), dual annihilation 511 keV photons, are emitted simultaneously from the body traveling in nearly opposite directions. Coincidence detection of these photons allows a line of response to be determined along which the radioactive decay event occurred. PET does not require a physical collimator for event localization. Nuclear images may be obtained using single photon emission (either planar or Single Photon Emission Computed Tomography (SPECT)) and Position Emission Tomography (PET). Planar imaging essentially compresses a three-dimensional radiation field onto a two-dimensional image plane, while SPECT and PET produce multiple image “slices,” each representing a different plane in of a three-dimensional region, such that when the slices are considered collectively, a three-dimensional image of the region may be studied.

Nuclear imaging is particularly suited to studying function and structure of tissue and organs, while other imaging modalities such as CT and MRI are more anatomically-oriented. Consequently, it would be particularly useful in oncological (e.g., tumor) studies to use SPECT or PET imaging to detect lesions, and to align or register the nuclear image with a medical image from another modality such as CT or MRI, which offers better anatomical information. Such a fused image would enable the clinician to determine the anatomical position of a lesion displayed by the nuclear image more accurately, and the organs and structures affected to be ascertained with higher accuracy and confidence.

Recently developed radiopharmaceutical tracers such as tracers based on monoclonal antibodies and labeled peptides have very high uptake in lesions or tumors and low uptake elsewhere. Thus, the tracer concentrates in the targeted tissue at such high levels that its resultant nuclear image manifests as a highly focused region of high intensity, with very little activity in other areas. Hence, the background region may contain little or no anatomical detail that would enable the high activity region to be localized with respect to the other structures or tissues of the patient's body. While such radiotracers thus are beneficial in the imaging of tumor metabolism, the lack of anatomical features in the nuclear image presents a problem in identifying the structures affected by the tumor or lesion using the nuclear image alone.

In recent years there has been considerable interest in development of techniques to co-register or align medical images of different modalities, such as PET and CT images, to thereby combine both functional and anatomical features in a single image. See, e.g., U.S. Pat. No. 6,490,476 to Townsend et al. In particular, techniques such as landmark registration or external marker registration are generally known in the art. Such techniques require either a significant amount of human interpretation of two separate images or require the use of external markers attached to the patient while two different imaging procedures are performed.

The '476 patent discloses the use of a combination CT and PET tomograph with a single patient bed, whereby sequential CT and PET images are obtained of a patient's region of interest and are displayed side-by-side on a monitor, or fused by interpolation of pixels from the two images. The '476 patent appears to rely on a fixed positional relationship between the CT scanner and the PET detector to effect alignment of the two images.

However, this requires that the two images be obtained simultaneously; patient movement during the relatively long imaging period can present a significant problem that can prevent accurate co-registration of the two images if images were obtained sequentially.

There remains a need in the art for improvement in co-registration and fusion of nuclear medical imaging data with conventional anatomical imaging data obtained with different modalities such as CT, MRI or US.

The concept of Compton scattering is well-known in the art, and is explained by D. B. Everett et al. in the paper entitled Gamma-radiation Imaging System Based On the Compton Effect, Proc. IEE, Vol. 124 (11), (1977), p. 995. Compton scatter occurs when a gamma photon radiates from a source along an incident path and collides with a particle at a point A on the incident path, whereupon it deposits a portion of its energy with the colliding particle, is scattered at an angle ⊖ and thereafter radiates along a scatter path in the direction ⊖.

The difference between the incident energy of the gamma photon and the scattered energy of the gamma photon is a measure of the scattering angle ⊖. The common expression of the Compton scattering formula computes the scattered energy as a function of the incident energy and the scattering angle ⊖ as follows:

$${E}_{sc} = \frac{{E}_{in}}{1 + \frac{{E}_{in}}{511 keV} ( 1 - cos Θ )} \qquad ( 1 )$$

wherein

E_{sc}=energy of scattered photon

Eᵢₙ=energy of incident photon

Consequently, scattered gamma photons entering the collimator of a conventional gamma camera will deposit a reduced amount of energy in the detector as compared with gamma photons emanating in a direct path from the source within the patient, and thus such scattered photons may be easily distinguished from unscattered photons. Since by definition the scattered photons have interacted with atoms or other particles at locations other than the location of the radiation source, the direction of such photons from the point of scatter may be inferred to a certain angle of uncertainty by making certain assumptions from the energy level of the detected scatter photon in the gamma camera detector.

### SUMMARY OF THE INVENTION

The present invention provides a novel system and method for more accurate co-registration or fusion of nuclear medical images with images obtained by other modalities such as CT, MRI or US, by acquiring and analyzing Compton scatter data coextensively with the acquisition of primary data such as SPECT photopeak data, and reconstructing Compton scatter images based on the acquired Compton scatter data to enhance anatomical surfaces or boundary regions in the SPECT images. The reconstructed Compton scatter images are then co-registered with the anatomical images obtained by CT, MRI or US to derive geometric transforms that are used to align or fuse the nuclear images with the anatomical modality images.

### BRIEF DESCRIPTION OF THE DRAWINGS

The present invention will become more fully understood from the detailed description given herein below and the accompanying drawings which are given by way of illustration only, and are not limitative of the present invention, and wherein:

FIG. 1 is a conceptual block diagram of a system for co-registering a nuclear medical image with an image of a different modality, according to a preferred embodiment of the present invention;

FIG. 2 is a diagram showing the use of the Compton scatter phenomenon to enhance anatomical boundary regions according to the present invention;

FIGS. 3A and 3B are diagrams illustrating an alternate embodiment of the invention wherein external radioactive sources are strategically placed adjacent to a subject of interest to enhance the Compton scatter phenomenon for use in obtaining increased anatomical boundary data; and

FIG. 4 is a diagram illustrating another alternate embodiment of the invention wherein scattered radiation from a second imaging apparatus such as a CT scanner is used to develop Compton scatter images that are subsequently co-registered with images from the second imaging apparatus.

### DETAILED DESCRIPTION OF THE PREFERRED EMBODIMENTS

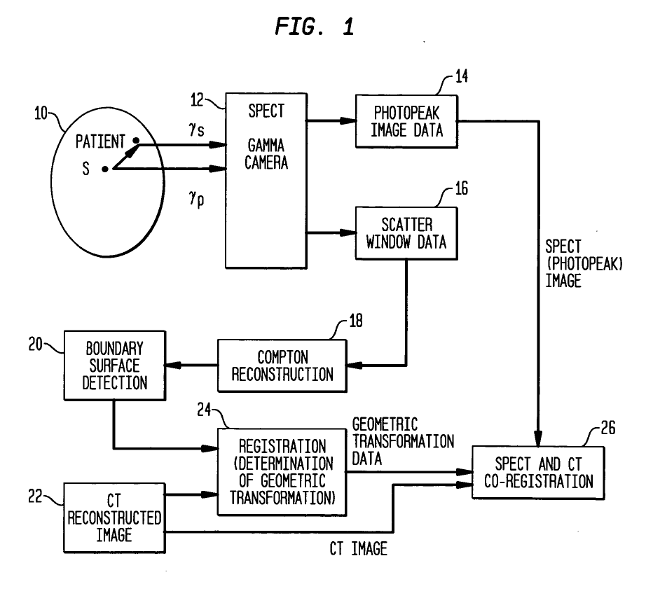

FIG. 1 is a block diagram showing the basic configuration of a co-registration system according to one embodiment of the invention. A conventional single photon gamma camera, such as SPECT gamma camera 12, is provided with two energy window discriminators: a photopeak discriminator that detects photopeak or unscattered gamma events Yₚ from a radiation source S within patient 10, and a Compton scatter discriminator that detects Compton scatter events Yₛ, which represent gamma photons that have collided with atomic particles at outer locations from the source S within the patient.

The accumulated photopeak image data 14 is reconstructed into a SPECT image and inputted to SPECT/CT co-registration processor 26. For purposes of simplicity, the present invention will be explained using the example of a SPECT image and a CT image; however, the invention is not so limited but may be applied to any form of nuclear medical image and form of other image modality, such as MRI and US imaging. Additionally, while each individual processing operation is described as a “processor” for simplicity of explanation, it will be recognized by those skilled in the art that the individual functions shown in FIG. 1 could be executed in many different ways, such as by software application modules running on a single microprocessor or other central processing unit, by separate microprocessors, by ASICs (Application Specific Integrated Circuits), by hardwired signal processing circuits (either digital or analog) or other types of electrical circuits.

The accumulated Compton scatter data 16 is reconstructed in Compton reconstruction processor 18 to form a Compton scatter image, which is inputted to anatomical boundary surface detection processor 20, which identifies and enhances anatomical boundary regions. The Compton scatter image reconstruction may use the same program and processor as the photopeak image reconstruction. The Compton scatter image contains information pertaining to the Compton coefficient μ_{c} and density of the tissue imaged. Gradient estimates of the Compton image may reveal the location of the body boundary, the boundary of large low density organs such as the lungs, or the boundaries of large, high density, tissue such as bones, etc., where a large gradient in pc and density exists. The gradient or “edge enhanced” reconstructed Compton image is obtained simultaneously with the SPECT photopeak image.

The Compton scatter image will contain boundary data for surface regions in the body where there is a large discontinuity of density or Compton attenuation coefficient μ_{c}. As shown in FIG. 2, a gamma photon emanating from source S within the body 200 of patient 10 in a direction toward lung 202 may scatter at the closer boundary of the lung, may enter the lung and scatter within the lung, may scatter at the farther boundary of the lung, or may pass through the lung and scatter at the boundary of the body 200. Stronger scatter gradients will exist at the boundaries of the lung and the body, while weaker scatter gradients will exist where the gamma photon scatters within the lung.

The reconstructed scatter projection data may be scaled and transformed in amplitude. A logarithmic transformation has been found to be useful. The resultant image can be surface or “edge” enhanced by filtering with a LaPlacian-like operator. The image then is co-registered in registration processor 24 with independently obtained anatomical image data such as CT reconstructed image 22, to obtain a geometric transform. The geometric transformation data then is inputted to the SPECT/CT co-registration processor 26, which also receives the SPECT photopeak image and the reconstructed CT image, and aligns or fuses the two images using the geometric transformation data. Alternately, the filtered Compton scatter image may be further refined to provide surface estimates.

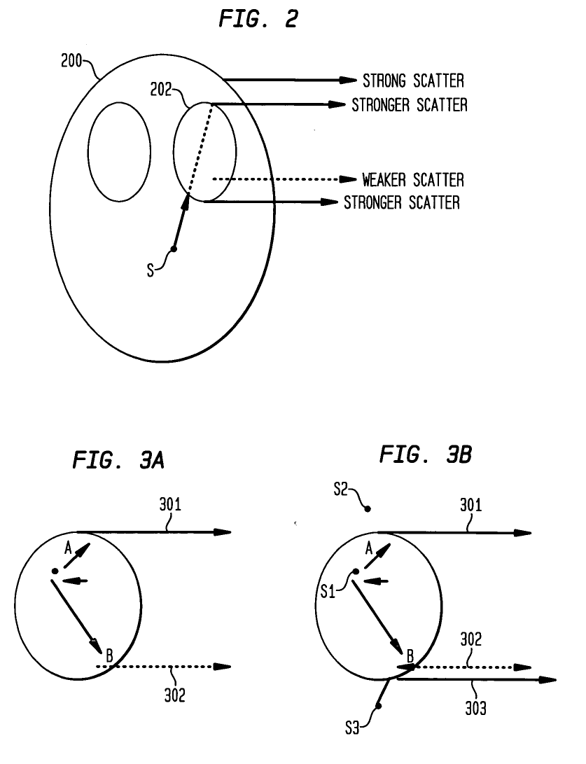

FIGS. 3A and 3B show an alternate embodiment of the invention, which may provide a benefit to certain particular imaging applications. As shown in FIG. 3A, where source S is closer to one boundary region of the patient than another, scatter 301 occurring at location A closer to the source S will have a stronger gradient than scatter 302 occurring at location B farther from the source S, which will be weaker.

As shown in FIG. 3B, strategically placing external radioisotope sources S2 and S3 adjacent to the patient may provide additional scattered photons at boundary locations farther from the internal radioisotope source S1 (in the example, at location B; however since it may not be known precisely where internal source S1 is located, two external sources are provided. If source S1 is capable of being localized, then the nearer external source S2 in the example of FIG. 3B may be eliminated.). As shown in FIG. 3B, external source S3 provides additional Compton scatter photons 303 to strengthen the scatter boundary data at the region B, farther from the internal source S1 than the region A.

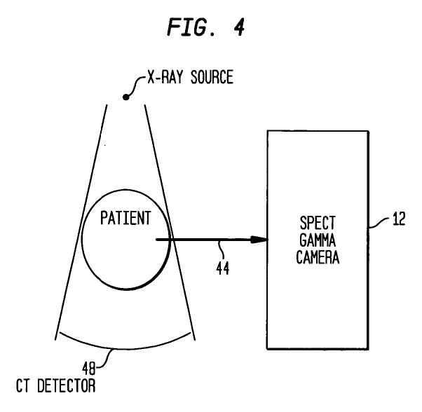

Another alternate embodiment of the invention is illustrated in FIG. 4. According to this embodiment, a gamma camera 12 and CT detector 48 are contained in the same system gantry (similar to the '476 patent discussed above). According to the present invention, the CT X-ray source may provide a scatter X-ray photon 44 for Compton scatter detection and imaging in the gamma camera 12.

Another possibility according to the invention would be to use a dual tracer technique, where the second tracer would be used to image anatomical features such as lungs and vasculature. The anatomical feature image then could be used for co-registration with the CT/MRI/US image. The second tracer need not be necessarily of a dose required to obtain a high quality anatomical image, but only sufficient for image co-registration purposes.

The invention having been thus described, it will be obvious to those skilled in the art that the same may be varied in many ways without departing from the spirit and scope of the invention. Any and all such modifications are intended to be included within the scope of the following claims.

What is claimed is:

1. A method for co-registering a nuclear medical image with a medical image of a different modality, comprising the steps of:
obtaining primary functional nuclear image data from a subject of interest;
obtaining secondary anatomical nuclear image data separate from said primary nuclear image data;
aligning said secondary anatomical nuclear image data with image data of said different modality, and determining a geometric transform required to align said anatomical image data and said different modality image data upon alignment of said image data; and
using said geometric transform to co-register said primary functional nuclear image data with said different modality image data.

2. The method of claim 1, wherein said secondary anatomical nuclear image data is Compton scatter data obtained from a radioactive tracer used to obtain said primary functional nuclear image data.

3. The method of claim 1, wherein said secondary anatomical nuclear image data is obtained from a radioactive tracer different than that used to obtain said primary functional nuclear image data.

4. The method of claim 2, further comprising the steps of using a scatter energy window to obtain said Compton scatter data and using a photopeak energy window to obtain said primary functional nuclear image data.

5. The method of claim 3, further comprising the steps of using a secondary energy window to obtain secondary anatomical nuclear image data different from a photopeak energy window to obtain said primary functional nuclear image data.

6. The method of claim 1, wherein said primary functional nuclear image data is SPECT data.

7. The method of claim 1, wherein said image data of said different modality is CT data.

8. The method of claim 1, wherein said image data of said different modality is MRI data.

9. The method of claim 1, wherein said image data of said different modality is ultrasound data.

10. The method of claim 2, further comprising the step of using a supplemental external source of Compton scatter photons to increase an amount of Compton scatter data obtained for use in reconstructing a secondary anatomical nuclear image.

11. The method of claim 10, wherein said supplemental external source of Compton scatter photons comprises a radioactive isotope.

12. The method of claim 10, wherein said supplemental external source of Compton scatter photons comprises an x-ray source used to obtain said different modality image.

13. Apparatus for co-registering a nuclear medical image with a medical image of a different modality, comprising:
a gamma camera that obtains primary functional nuclear image data from a subject of interest, and obtains secondary anatomical nuclear image data separate from said primary nuclear image data;
means for aligning said secondary anatomical nuclear image data with image data of said different modality, and determining a geometric transform required to align said anatomical image data and said different modality image data upon alignment of said image data; and
means for using said geometric transform to co-register said primary functional nuclear image data with said different modality image data.

14. The apparatus of claim 13, wherein said secondary anatomical nuclear image data is Compton scatter data obtained from a radioactive tracer used to obtain said primary functional nuclear image data.

15. The apparatus of claim 13, wherein said secondary anatomical nuclear image data is obtained from a radioactive tracer different than that used to obtain said primary functional nuclear image data.

16. The apparatus of claim 14, further comprising a scatter energy window that obtains said Compton scatter data and using a photopeak energy window to obtain said primary functional nuclear image data.

17. The apparatus of claim 15, further comprising a secondary energy window that obtains secondary anatomical nuclear image data different from a photopeak energy window to obtain said primary functional nuclear image data.

18. The apparatus of claim 13, wherein said primary functional nuclear image data is SPECT data.

19. The apparatus of claim 13, wherein said image data of said different modality is CT data.

20. The apparatus of claim 13, wherein said image data of said different modality is MRI data.

21. The apparatus of claim 13, wherein said image data of said different modality is ultrasound data.

22. The apparatus of claim 14, further comprising a supplemental external source of Compton scatter photons to increase an amount of Compton scatter data obtained for use in reconstructing a secondary anatomical nuclear image.

23. The apparatus of claim 22, wherein said supplemental external source of Compton scatter photons comprises a radioactive isotope.

24. The apparatus of claim 22, wherein said supplemental external source of Compton scatter photons comprises an x-ray source used to obtain said different modality image.
